(19) United States
(12) Patent Application Publication
(10) Pub. No.: US 2002/0161263 A1
(43) Pub. Date: Oct. 31, 2002

(54) Process for the preparation of ketones

(75) Inventors: Nongyuan Shi, Hanau (DE); Steffen Krill, Speyer (DE); Klaus Huthmacher, Gelnhausen (DE); Bernd Drapal, Alzenau (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: DEGUSSA AG, Duesseldorf (DE)

(21) Appl. No.: 10/132,196

(22) Filed: Apr. 26, 2002

(30) Foreign Application Priority Data
Apr. 28, 2001 (DE) 101 21 057.4

(51) Int. Cl. C07C 45/68

(52) U.S. Cl. 568/343; 568/391

(57) ABSTRACT

A process for the preparation of ketones corresponding to the general Formula I

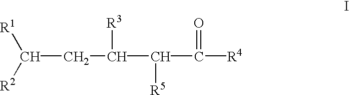

in which β,γ,δ-allenic, di-unsaturated ketones and/or α,β,γ,δ-conjugated, di-unsaturated ketones, obtained by reaction of propargyl alcohols with enol ethers and are converted in direct manner by hydrogenation to the saturated ketones corresponding to the general Formula I without purification by distillation.

### BACKGROUND OF THE INVENTION

1. Field of the Invention

The present invention relates to an improved process for the preparation of ketones by a two-stage reaction of propargyl alcohols with enol ethers to obtain β,γ,δ-allenic and/or α,β,γ,δ-conjugated, di-unsaturated ketones, followed by the direct hydrogenation to the unsaturated ketones.

2. Discussion of the Background

A reaction between a propargyl alcohol and an enol ether was described for the first time by Marbet and Saucy in Chimia 14 (1960), pages 362 to 363.

DE 1 230 783 describes a process for the preparation of polyene ketones and their isomerization products with secondary alcohols in the presence of acid catalysts such as, for example, sulfuric or phosphoric acid.

The U.S. Pat. No. 3,029,287 and the publication by R. Marbet and G. Saucy, Helv. Chim. Acta (1967) 50, 1158-1167 describe a process for the preparation of β,γ,δ-unsaturated ketones by the reaction of propargyl alcohols with enol ethers in the presence of an acid catalyst.

It was described in DE 199 49 796.6 that the Saucy-Marbet reaction is catalyzed efficiently by aliphatic sulfonic acids or sulfonic acid salts.

The β,γ,δ-allenic and/or α,β,γ,δ-conjugated, di-unsaturated ketones according to the invention are normally obtained first by distillation, and then converted by hydrogenation to saturated ketones corresponding to the general Formula I. Since these β,γ,δ-allenic and/or α,β,γ,δ-conjugated, di-unsaturated ketones are not thermally stable, the valuable β,γ,δ-allenic and/or α,β,γ,δ-conjugated, di-unsaturated ketones are lost during purification by distillation.

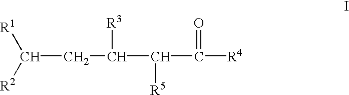

The prior art describes no process for the preparation of ketones by the reaction of an unsaturated alcohol and an enol ether in a Saucy-Marbet reaction to form saturated ketones, followed by the direct hydrogenation of these unsaturated ketones.

### SUMMARY OF THE INVENTION

Accordingly, an object of the invention is therefore to provide a process for the reaction between propargyl alcohols and enol ethers to obtain β,γ,δ-allenic and/or α,β,γ,δ-conjugated, di-unsaturated ketones and subsequent hydrogenation, so as to enable the hydrogenation of the unsaturated ketones to take place in direct manner following their preparation, without prior treatment/purification of these unsaturated ketones by distillation.

### DESCRIPTION OF THE PREFERRED EMBODIMENTS

The present invention relates to a process for the preparation of ketones corresponding to the general Formula I

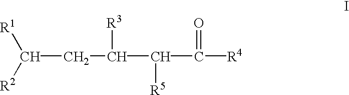

in which

R¹ and R² are hydrogen, a saturated C₁-C₂₀ alkyl radical which is optionally substituted with oxygen-containing groups and may be branched or unbranched, or a C₁-C₂₀ alkylaryl radical, whereby the radicals R¹ and R² may also together form a 5- or 6-membered ring;

R³ and R⁵ are hydrogen or a C₁ to C₄-alkyl radical; and

R⁴ is a hydrogen or a C₁ to C₄ alkyl radical;

and/or mixture thereof, by a two-stage reaction which comprises the following steps:

(1) reaction, in the presence of an acid catalyst, of propargyl alcohols corresponding to the general Formula II

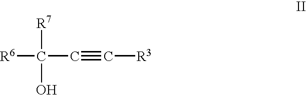

in which

R⁶ and R⁷ are hydrogen, a C₁-C₂₀ alkyl radical which is optionally substituted with oxygen-containing groups and may be saturated or unsaturated, branched or unbranched, or a C₁-C₂₀ alkylaryl radical, whereby the radicals R¹ and R² may also together form a 5- or 6-membered ring,

R³ denotes the same as indicated above, with enol ethers corresponding to the general Formula III

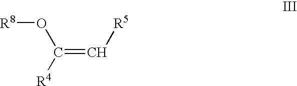

in which

R⁴ and R⁵ denote the same as indicated above, and

R⁸ is a C₁- to C₄-alkyl radical, preferably a methyl radical or ethyl radical; to obtain a mixture of β,γ,δ-allenic, di-unsaturated ketones corresponding to the general Formula IV A and α,β,γ,δ-conjugated, di-unsaturated ketones corresponding to the general Formula IV B

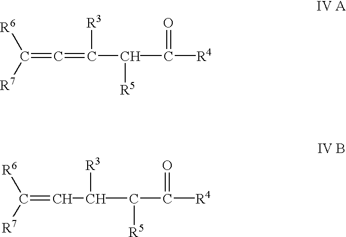

in which R³, R⁴, R⁵, R⁶ and R⁷ denote the same as indicated above, followed by hydrogenation of the mixture of β,γ,δ-allenic, di-unsaturated ketones corresponding to the general Formula IV A and α,β,γ,δ-conjugated, di-unsaturated ketones corresponding to the general Formula IV B, which is described above, in the presence of a noble metal catalyst.

A range of ketones corresponding to the general Formula I constitute valuable intermediate products for the preparation of vitamin E.

The process according to the invention is characterized in that β,γ,δ-allenic, di-unsaturated ketones corresponding to the general Formula IV A and/or α,β,γ,δ-conjugated, di-unsaturated ketones corresponding to the general Formula IV B, which are obtained from propargyl alcohols corresponding to the general Formula II with enol ethers corresponding to the general Formula III, can be converted in direct manner by hydrogenation to saturated ketones corresponding to the general Formula I without purification by distillation.

When the reaction is carried out in accordance with the process according to the invention, not only are some energy-intensive steps avoided, but also, as a result of dispensing with the distillation of the thermolabile allene ketone and dienone intermediate products, the total yield of saturated ketones is increased by approximately 10% in relation to propargyl alcohol.

Preferred propargyl alcohols corresponding to the Formula II in the process according to the invention are those in which

R⁶ stands for a C₁-C₂₀ alkyl radical which may be saturated or unsaturated, branched or unbranched, a C₁-C₂₀-aryl radical, or an arylalkyl radical,

R⁷ stands for a C₁ to C₄ alkyl radical, in particular a methyl radical, and

R³ stands for hydrogen or a C₁ to C₄-alkyl radical; preferably hydrogen.

The following are examples of suitable propargyl alcohols:

3-methyl-1-butyn-3-ol;

3,7-dimethyl-6-octen-1-yn-3-ol (dehydrolinalool);

3,7-dimethyl-5-octen-1-yn-3-ol;

3,7-dimethyl-4-octen-1-yn-3-ol;

3,7-dimethyl-1-octyn-3-ol (hydrodehydrolinalool);

3,7,11-trimethyl-6,10-dodecadien-1-yn-3-ol (dehydronerolidol);

3,7,11-trimethyl-6-dodecen-1-yn-3-ol;

3,7,11-trimethyl-1-dodecyn-3-ol (hydrodehydronerolidol);

1-ethynyl-1-cyclohexanol; and

1-ethynyl-2,2,6-trimethyl-1-cyclohexanol.

As enol ethers corresponding to the Formula III, compounds in which

R⁴ stands for a methyl radical or ethyl radical,

R⁵ stands for hydrogen or a methyl radical, and

R⁸ stands for a methyl radical or ethyl radical are preferably considered.

The following might examples of suitable enol ethers: isopropenyl methyl ether, isopropenyl ethyl ether, isopropenyl propyl ether, isopropenyl butyl ether, isopropenyl isobutyl ether, 2-methoxy-1-butene, 2-ethoxy-1-butene, 2-propoxy-1-butene, 3-butoxy-1-butene, 2-methoxy-2-butene, 2-ethoxy-2-butene, 2-methoxy-1-pentene, 2-ethoxy-1-pentene, 2-methoxy-2-pentene, 2-ethoxy-2-pentene, 3-methoxy-3-pentene, 3-ethoxy-2-pentene, in particular isopropenyl methyl ether.

On the industrial scale isopropenyl methyl ether is frequently preferred for reasons relating to both economics and process engineering. The dimethoxypropane formed from it can be readily recovered by distillation from the reaction mixture and re-utilized for the preparation of isopropenyl methyl ether.

The first reaction step takes place at temperatures of between approximately 50° C. and 200° C., preferably between 60° C. and 170° C., particularly preferably between 80° C. and 130° C. A particularly high reaction rate such as, for example, less than 5 hours, preferably less than 4 hours, particularly preferably less than 3 hours, is obtained with no observable impairment of the selectivity, when the reaction is carried out at different temperature levels which are adjusted dependent on the degree of conversion of the unsaturated alcohol. At the beginning of the reaction a temperature is normally adjusted, which is approximately 10° C.-30° C. lower than the temperature level at the end of the reaction.

The reaction may be carried out in a batch, semi-batch or continuous process. The reaction may furthermore be carried out in pressureless manner but also under pressure. In the case of a pressure reaction the reaction takes place within the pressure range 1 to 20 bar, preferably 1 to 10 bar.

The molar ratio between the propargyl alcohol corresponding to the Formula II and the enol ether corresponding to the Formula III in the process according to the invention is generally between 1:2 and 1:10, preferably 1:2.05 to 1:5, particularly preferably 1:2.05 to 1:3.5. The excess enol ether may be recovered by distillation after the reaction has ended.

The following serve as acid catalysts of the first reaction step in the process according to the invention: mineral acids such as, for example, sulfuric or phosphoric acid and salts thereof, strong organic acids such as oxalic acid, trichloroacetic acid, p-toluic acid, as well as Lewis acids such as zinc chloride or boron trifluoride etherate, and aliphatic sulfonic acids and salts of the corresponding sulfonic acids having acid properties are particularly preferred.

The following are examples of suitable aliphatic sulfonic acids: methanesulfonic acid, ethanesulfonic acid, propanesulfonic acid, butanesulfonic acid, pentanesulfonic acid, hexanesulfonic acid, chloromethanesulfonic acid, in particular methanesulfonic and ethanesulfonic acid.

The following are examples of suitable sulfonic acid salts: pyridinium p-toluolsolfonate, tetramethylammonium p-toluenesulfonate, pyridinium methanesulfonate, pyridinium ethanesulfonate, in particular pyridinium p-toluolsolfonate and pyridinium methanesulfonate, which is optionally able to form “in situ” from the corresponding acid and the corresponding base.

A solvent such as acetone, methyl isobutyl ketone, methyl isopropyl ketone, ethanoic acid, formic acid, propionic acid, 2-ethylhexanoic acid may be used as a solvent for the acid catalysts. It is, however, also possible to utilize the unsaturated alcohol corresponding to the general Formula II, in which R1 and R2 denote the same as indicated above, which was utilized as an educt.

The reaction in the first process step may be carried out with or without reaction solvent. Reactions which are carried out in solvent-free manner are preferred. The following may be utilized as suitable reaction solvents within the scope of the present invention: hydrocarbons, for example hexane, heptane, octane, toluene and xylene; and ketones, for example isobutyl methyl ketone, diethyl ketone and isophorone, dimethoxypropane.

A cascade of stirred-tank reactors or tubular reactors designed for reactions under pressure, or a cascade of corresponding stirred-tank reactors and tubular reactors is can be used as the reaction vessel.

The subsequent hydrogenation is carried out in the presence of a noble metal catalyst and under pressure. The temperature of the hydrogenation is between room temperature (approx. 25° C.) and 100° C., preferably room temperature (approx. 25° C.) to 80° C. Raney nickel, Pd/C or Pt/C compounds may be used as a hydrogenation catalyst. The Pd/C compound is a particularly preferred hydrogenation catalyst. The quantity of catalyst is from 0.3% to 5%, preferably 0.5% to 1%, in relation to the unsaturated ketones which are to be hydrogenated.

The Examples which follow are intended to illustrate specific embodiments of the invention and are not intended to further limit the invention.

### EXAMPLE 1

Preparation of phytone from 3,7,1 1-trimethyl-1--dodecin-3-ol

89.8 grams (g) 3,7,11-trimethyl-1-dodecin-3-ol (0.4 mole) and 101 g isopropenyl methyl ether (1.4 mole) are charged into a pressure vessel. The reactor is purged with nitrogen and the pressure raised to 2 bar. The educt mixture is heated to 110° C. A solution of 104 milligrams (mg) methanesulfonic acid dissolved in 45 milliliters (ml) acetone is dispensed-in portion-wise by a pump within 4 hours (h). After a total of 4.5 h 3,7,11-trimethyl-1-dodecin-3-ol is completely reacted. The autoclave is cooled to room temperature (approx. 25° C.), and the pressure is released.

The reaction mixture is neutralized by the addition of a methanolic NaOAc solution. Low-boiling components, primarily excess isopropenyl methyl ether and 2,2-dimethoxypropane, are then separated on a rotary film evaporator and condensed in a cold trap. The mixture of 6,10,14-trimethylpentanedeca-4,5-dien-2-one and 6,10,14-trimethylpentanedeca-3,5-dien-2-one, which is obtained, is hydrogenated to phytone with Pd/C catalyst. 99 g phytone are obtained, which corresponds to a total yield of 93% in relation to 3,7,1 1-trimethyl-1-dodecin-3-ol.

Comparative Example A

Preparation of phytone from 3,7,1 1-trimethyl-1--dodecin-3-ol

1) 103 g 3,7,11-trimethyl-1-dodecin-3-ol (0.46 mole) and 99.5 g isopropenyl methyl ether (1.38 mole) are charged into a pressure vessel. The reactor is purged with nitrogen and the pressure raised to 2 bar. The educt mixture is heated to 90° C. A solution of 76 mg methanesulfonic acid in 60 ml acetone is added to this within 90 min. The mixture is then stirred at 115° C. for a further hour. 98% conversion of 3,7,1 1-trimethyl-1-dodecin-3-ol is obtained. The autoclave is cooled to room temperature, and the pressure is released. The reaction mixture is neutralized by the addition of a methanolic NaOAc solution. Low-boiling components, primarily excess isopropenyl methyl ether and 2,2-dimethoxypropane, are then separated on a rotary film evaporator and condensed in a cold trap. 128 g residue of 6,10,14-trimethylpentanedeca-4,5-dien-2-one and 6,10,14-trimethylpentanedeca-3,5-dien-2-one and by-products are obtained. The residue is distilled under vacuum, with 103 g of a mixture of 6,10,14-trimethylpentanedeca-4,5-dien-2-one and 6,10,14-trimethylpentanedeca-3,5-dien-2-one being obtained. This corresponds to a yield of 85% in relation to 3,7,1 1-trimethyl-1-dodecin-3-ol.

2) 80 g of the mixture of 6,10,14-trimethylpentanedeca-4,5-dien-2-one and 6,10,14-trimethylpentanedeca-3,5-dien-2-one obtained in 1) are dissolved in 200 ml isopropanol, to this is added 0.8 g 10% Pd/C catalyst. The mixture is hydrogenated at 5 bar and 40° C. 76 g phytone are obtained following the removal of the solvent, this corresponds to a yield of 94% in relation to the unsaturated ketone.

The total phytone yield in relation to 3,7,11-trimethyl-1-dodecin-3-ol is 80%.

### EXAMPLE 2

Preparation of phytone from 3,7,11-trimethyl--dodec-1-yn-3-ol

56.1 g 3,7,11-trimethyl-dodec-1-yn-3-ol (0.25 mole) and 63.1 g isopropenyl methyl ether (0.875 mole) are charged into a nitrogen-purged pressure vessel. The reactor is closed, and the pressure is raised with nitrogen to 2 bar. The educt mixture is heated to 95° C. A solution of 58 mg methanesulfonic acid in 7 ml acetone is dispensed-in portion-wise by a pump within 2.5 hours (h). The reaction is then held at 110° C. for a further 30 minutes (min). After a total of 3 h 3,7,11-trimethyl-dodec-1-yn-3-ol is 99% reacted.

The autoclave is cooled to room temperature (approx. 25° C.), and the pressure is released. The reaction mixture is neutralized by the addition of a methanolic sodium acetate solution. Low-boiling components, primarily excess isopropenyl methyl ether and 2,2-dimethoxypropane, are then separated on a rotary film evaporator and condensed in a cold trap. The mixture of 6,10,14-trimethyl-penta-deca-4,5-dien-2-one and 6,10,14-2tp trimethylpentanedeca-3,5-dien-2-one, which is obtained, is hydrogenated in 2-propanol to phytone with a Pd/C catalyst. 62 g phytone are obtained following the removal of the solvent. This corresponds to a total yield of 93% in relation to 3,7,11-trimethyl-dodec-1-yn-3-ol.

### EXAMPLE 3

Preparation of phytone from 3,7,11-trimethyl--dodec-1-yn-3-ol

As described in Example 1, 67.3 g 3,7,11-trimethyl-dodec-1-yn-3-ol (0.30 mole) and 75.7 g isopropenyl methyl ether (1.05 mole) are charged into a nitrogen-purged pressure vessel. The reactor is closed, and the pressure is raised with nitrogen to 2 bar. The educt mixture is heated to 80° C. A solution of 80 mg methanesulfonic acid in 10 ml acetone is dispensed-in portion-wise by a pump within 3 h. 3,7,11-trimethyl-dodec-1-yn-3-ol is 99% converted.

The autoclave is cooled to room temperature (approx. 25° C.), and the pressure is released. The reaction mixture is neutralized by the addition of a methanolic sodium acetate solution. Low-boiling components, primarily excess isopropenyl methyl ether and 2,2-dimethoxypropane, are then separated on a rotary film evaporator and condensed in a cold trap. The mixture of 6,10,14-trimethyl-pentadeca-4,5-dien-2-one and 6,10,14-trimethyl-penta-deca-3,5-dien-2-one, which is obtained, is hydrogenated in 2-propanol to phytone with a Pd/C catalyst. 74 g phytone result following the removal of the reaction solvent. This corresponds to a total yield of 92% in relation to 3,7,11-trimethyl-dodec-1-yn-3-ol.

### EXAMPLE 4

Preparation of phytone from 3,7,11-trimethyl--dodec-1-yn-3-ol

94.3 g 3,7,11-trimethyl-dodec-1-yn-3-ol (0.42 mole) and 90.9 g isopropenyl methyl ether (1.26 mole) are charged into a pressure vessel under nitrogen. The reactor is closed, and the pressure is raised with nitrogen to 2 bar. The educt mixture is heated to 90° C. A solution of 97 mg methanesulfonic acid in 12 ml acetone is dispensed-in portion-wise by a pump within 3 h. In the first hour the temperature is held at between 95° C. and 100° C., and then heated to 115° C. In parallel with the heating 16.1 g isopropenyl methyl ether (0.23 mole) are dispensed-in by way of a pump within 30 minutes (min). Stirring of the mixture continues for 30 min. The 3,7,11-trimethyl-dodec-1-yn-3-ol conversion is around 99%.

Cooling takes place to room temperature (approx. 25° C.), and the pressure is ma released. The reaction mixture is neutralized by the addition of a methanolic sodium acetate solution. Low-boiling components, primarily excess isopropenyl methyl ether and 2,2-dimethoxypropane, are then separated on a rotary film evaporator and condensed in a cold trap. The mixture of 6,10,14-trimethyl-penta-deca-4,5-dien-2-one and 6,10,14-trimethyl-pentadeca-3,5-dien-2-one, which is obtained, is hydrogenated in 2-propanol to phytone with a Pd/C catalyst. 105 g phytone are obtained following the removal of the reaction solvent. This corresponds to a total yield of 93% in relation to 3,7,11-trimethyl-dodec-1-yn-3-ol.

### EXAMPLE 5

Preparation of phytone from 3,7,11-trimethyl--dodec-1-yn-3-ol

As described in Example 3, 89.8 g 3,7,11-trimethyl-dodec-1-yn-3-ol (0.40 mole) and 57.7 g isopropenyl methyl ether (0.80 mole) are charged into a pressure vessel. The reactor is closed, and the pressure is raised with nitrogen to 2 bar. The educt mixture is heated to 95° C. A solution of 66 mg methanesulfonic acid in 8 ml acetone is dispensed-in portion-wise by a pump within 2.5 h. In the first 45 minutes the temperature is held at between 95 and 100° C. and then heated to 115° C. In parallel with the heating 14.4 g isopropenyl methyl ether (0.20 mole) are dispensed-in within 20 minutes. The 3,7,11-trimethyl-dodec-1-yn-3-ol conversion is around 99%.

Cooling takes place to room temperature (approx. 25° C.), and the pressure is released. The reaction mixture is neutralized by the addition of a methanolic sodium acetate solution. Low-boiling components, primarily excess isopropenyl methyl ether and 2,2-dimethoxypropane, are then separated on a rotary film evaporator and condensed in a cold trap. The mixture of 6,10,14-trimethyl-penta-deca-4,5-dien-2-one and 6,10,14-trimethyl-penta-deca-3,5-dien-2-one, which is obtained, is hydrogenated in 2-propanol to phytone with Pd/C catalyst. 99.2 g phytone are obtained. This corresponds to a total yield of 93% in relation to 3,7,11-trimethyl-dodec-1-yn-3-ol.

### EXAMPLE 6

Preparation of tetrahydrogeranyl acetone from 3,7-dimethyl-oct-1-yn-3-ol 231.4 g 3,7-dimethyl-oct-1-yn-3-ol (1.50 mole) and 324.5 g isopropenyl methyl ether (4.50 mole) are charged into a pressure vessel under nitrogen. The reactor is closed, and the pressure is raised with nitrogen to 2 bar. The educt mixture is heated to approx. 90° C. A solution of 265 mg methanesulfonic acid in 16 ml acetone is dispensed-in portion-wise by a pump within 1.5 h. Stirring is continued for 30 min at 90-92° C. The 3,7-dimethyl-oct-1-yn-3-ol conversion is around 99%.

The mixture is cooled to room temperature (approx. 25° C.), and the pressure is released. The reaction mixture is neutralized by the addition of a small quantity of triethylamine. Low-boiling components, primarily excess isopropenyl methyl ether and 2,2-dimethoxypropane, are then separated on a rotary film evaporator and condensed in a cold trap. The mixture of 6,10-dimethyl-undeca-4,5-dien-2-one and 6,10-dimethyl-undeca-3,5-dien-2-one, which is obtained, is hydrogenated in 2-propanol to tetrahydrogeranyl acetone with a Pd/C catalyst. 271 g tetrahydrogeranyl acetone are obtained. This corresponds to a total yield of 91% in relation to 3,7-dimethyl-oct-1-yn-3-ol.

### EXAMPLE 7

Preparation of tetrahydrogeranyl acetone from 3,7-dimethyl-oct-1-yn-3-ol

216.0 g 3,7-dimethyl-oct-1-yn-3-ol (1.40 mole) and 324.5 g isopropenyl methyl ether (4.50 mole) are charged into a pressure vessel under nitrogen. The reactor is closed, and the pressure is raised with nitrogen to 2 bar. The educt mixture is heated to 85° C. A solution of 234 mg methanesulfonic acid in 16 ml 3,7-dimethyl-oct-1-yn-3-ol is dispensed-in portion-wise by a pump within 1.5 h. Stirring is continued for 30 min at 90-92° C. The 3,7-dimethyl-oct-1-yn-3-ol conversion is around 99%.

Cooling takes place to room temperature (approx. 25° C.), and the pressure is released. The reaction mixture is neutralized by the addition of 3.0 ml of a methanolic sodium acetate solution (0.10 g/ml). Low-boiling components, primarily excess isopropenyl methyl ether and 2,2-dimethoxypropane, are then separated on a rotary film evaporator and condensed in a cold trap. The mixture of 6,10-dimethyl-undeca-4,5-dien-2-one and 6,10-dimethyl-undeca-3,5-dien-2-one, which is obtained, is hydrogenated in 2-propanol to tetrahydrogeranyl acetone with Pd/C catalyst. 273 g tetrahydrogeranyl acetone are obtained. This corresponds to a total yield of 92% in relation to 3,7-dimethyl-oct-1-yn-3-ol.

### EXAMPLE 8

Preparation of tetrahydrogeranyl acetone from 3,7-dimethyl-oct-1-yn-3-ol

77.2 g 3,7-dimethyl-oct-1-yn-3-ol (0.50 mole) and 108.2 g isopropenyl methyl ether (1.50 mole) are charged into a pressure vessel under nitrogen. The reactor is closed, and the pressure is raised with nitrogen to 2 bar. The educt mixture is heated to 90° C. 9.0 ml of a solution of 403 mg sulfuric acid in 50 ml acetone are dispensed-in portion-wise within 1.5 h. After continued stirring for 1.5 h at 90° C. to 95° C. a conversion of 99% is obtained.

The autoclave is cooled to room temperature, and the pressure is released. The reaction mixture is neutralized by the addition of 1.5 ml of a methanolic sodium acetate solution (0.10 g/ml). Low-boiling components, primarily excess isopropenyl methyl ether and 2,2-dimethoxypropane, are then separated on a rotary film evaporator and condensed in a cold trap. The mixture of 6,10-dimethyl-undeca-4,5-dien-2-one and 6,10-dimethyl-undeca-3,5-dien-2-one, which is obtained, is hydrogenated in 2-propanol to tetrahydrogeranyl acetone with a Pd/C catalyst. The solvent is separated on a rotary film evaporator and condensed in a cold trap. 90 g tetrahydrogeranyl acetone are obtained. This corresponds to a total yield of 91% in relation to 3,7-dimethyl-oct-1-yn-3-ol.

Obviously, numerous modifications and variations of the present invention are possible in light of the above teachings. It is therefore to be understood that within the scope of the appended claims, the invention may be practiced otherwise than as specifically described herein.

German Application 10121057.4, filed on Apr. 28, 2001, is incorporated by reference in its entirety.

1. A process for the preparation of one or more ketones corresponding to Formula I 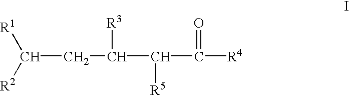
in which
R¹ and R² may be the same or different and each may be hydrogen, a saturated C₁-C₂₀ alkyl radical which may be substituted with oxygen-containing groups and may be branched or unbranched, or a C₁-C₂₀ alkylaryl radical, wherein the radicals R¹ and R² may form a 5- or 6-membered ring;
R³ and R⁵ may be the same or different and each may be hydrogen or a C₁ to C₄-alkyl radical; and
R⁴ may be a hydrogen or a C₁ to C₄ alkyl radical; by a two-stage reaction which comprises:
(1) reacting at least one propargyl alcohol corresponding to Formula II in the presence of an acid catalyst, 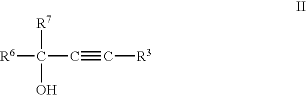
in which
R⁶ and R⁷ may be the same or different and each may be hydrogen, a C₁-C₂₀ alkyl radical which may be substituted with oxygen-containing groups and may be saturated or unsaturated, branched or unbranched, or a C₁-C₂₀ alkylaryl radical, wherein the radicals R⁶ and R⁷ may form a 5- or 6-membered ring, with at least one enol ether corresponding to Formula III 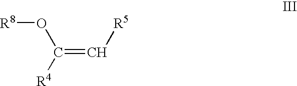
in which
R⁸ may be a C₁- to C₄-alkyl radical; to obtain a mixture comprising one or more β,γ,δ-allenic, di-unsaturated ketones corresponding to Formula IV A and one or more α,β,γ,δ-conjugated, di-unsaturated ketones corresponding to Formula IV B 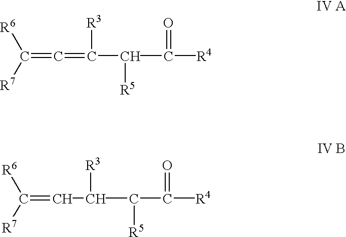
followed by
(2) hydrogenating the mixture of β,γ,δ-allenic, di-unsaturated ketones and α,β,γ,δ-conjugated, di-unsaturated ketones in the presence of a noble metal catalyst;
wherein the di-unsaturated ketones may be hydrogenated to one or more ketones of Formula I without purification by distillation.

2. The process according to claim 1, wherein the hydrogenation temperature is between room temperature and 100° C.

3. The process according to claim 2, wherein the hydrogenation temperature is between room temperature and 80° C.

4. The process according to claim 1, wherein the noble metal catalyst is a Raney nickel, Pd/C or Pt/C compound.

5. The process according to claim 4, wherein the noble metal catalyst is a Pd/C compound.

6. The process according to claim 1, wherein the catalyst is selected from the group consisting of a mineral acid, a Lewis acid, an aliphatic sulfonic acid, an organic acid, salts thereof, and mixtures thereof.

7. The process as claimed in claim 1, wherein the reaction is carried out in the absence of a solvent.

8. The process of claim 1, wherein
R² is a methyl group,
R³is hydrogen,
R⁴ is methyl or ethyl,
R⁵ is hydrogen or methyl, and
R⁸ is methyl or ethyl.

9. The process as claimed in claim 1 wherein a ratio of the propargyl alcohol to the enol ether is from 1:2.05 to 1:3.5.

10. A composition comprising the mixture of di-unsaturated ketones obtained by the process of claim 1.

11. The process according to claim 1, wherein the ketone of Formula I is phytone, the propargyl alcohol of Formula II is 3,7,11-trimethyl-l-dodecin-3-ol, and the enol ether is isopropenylmethyl ether.

12. The process according to claim 1, wherein the ketone of Formula I is phytone, the propargyl alcohol of Formula II is 3,7,1 -trimethyldodec-1-yn-3-ol, and the enol ether of Formula III is isopropenyl methyl ether.

13. The process according to claim 1, wherein the ketone of Formula I is tetrahydrogeranyl acetone, the propargyl alcohol of Formula II is 3,7-dimethyl-oct-1-yn-3-ol, and the enol ether of Formula III is isopropenylmethyl ether.

14. The process as claimed in claim 1, wherein the acid catalyst is added portion-wise during the reaction.

15. The process according to claim 1, further comprising removing low boiling components after reaction and before hydrogenation.
